(19) United States
(12) Patent Application Publication
(10) Pub. No.: US 2006/0089407 A1
(43) Pub. Date: Apr. 27, 2006

(54) Methods for Making and Using Synergistic Multifunctional Compositions

(75) Inventor: Gerald L. Maurer, Cincinnati, OH (US)

Correspondence Address: LAFKAS PATENT LLC, 7811 LAUREL AVENUE, CINCINNATI, OH, 45243, US

(73) Assignee: NATIONAL RESEARCH LABORATORIES, LTD., Cincinnati, OH (US)

(21) Appl. No.: 11/163,624

(22) Filed: Oct. 25, 2005

Related U.S. Application Data
(60) Provisional application No. 60/522,648, filed on Oct. 25, 2004.

(51) Int. Cl. A61K 31/315; A61K 31/30

(52) U.S. Cl. 514/494; 514/499

(57) ABSTRACT

The present invention is a synergistic palliative and/or therapeutic admixture for treating and/or medicating affected biological tissue in mammals. The admixture includes two or more multivalent metals and at least one polyfunctional organic ligand. The ligand is in the form of an alkaline earth salt. The molar ratio of metal to ligand is 1:1.

### CROSS-REFERENCE TO RELATED APPLICATION

This application for a patent claims priority to U.S. Provisional Patent Application No. 60/522,648 as filed Oct. 25, 2004.

### BACKGROUND

The various exemplary embodiments of the present invention relate generally to a composition and method of using the composition to palliate or treat affected biological tissues in mammals. More particularly, the various exemplary embodiments of the present invention relate to a method and a composition for treating damaged biological tissue comprising two or more synergistically combined monometal complexes of multivalent metals with a polyfunctional organic ligand.

Inflammation is a local and protective response to tissue injury and destruction of cells. The precise elements constituting the inflammatory response vary according to the site of injury, the state of the body, and the injurious agent, such as bacteria or trauma. Should the inflammatory response become impaired or compromised, however, the corresponding tissue will undergo a degenerative process stimulating further injury and cell destruction. Obviously, then, the inflammatory response embodies a multifaceted process that is required to promote and rehabilitate normal tissue function. Therefore, since the inflammatory response is generally similar with various stimuli, it can be viewed and treated as a relatively nonspecific response.

Presently, conventional anti-inflammatory therapy includes application of heat, exercise, salicylates to tolerance, indomethacin or butazolidin, and oral and intra-articular steroids. The above anti-inflammatory protocol, however, is less than optimum because it provides only a means to inhibit some component of the inflammatory process in a generally temporary or transient fashion. In other words, it treats the symptoms rather than promoting tissue repair or alleviating the causes of the degeneration.

Currently there are known methods of treating inflammation of tissue with metals such as copper. For example, it has been known since ancient Egypt that copper has been indicated for therapeutically treating granulomatous inflammation. It has been well established that the dissolution of copper from copper jewelry, for example, bracelets, worn in contact with skin appears to have therapeutic anti-inflammatory effects. In other studies, subdermal copper implants in rats have been demonstrated to exhibit anti-inflammatory activity. In a further instance, a neutral copper (II) bis(glycine) complex perfused through cat skin demonstrating that skin is permeable to soluble copper. In still a further instance several oral and parenteral copper complexes have been somewhat successfully used in the treatment of inflammation or arthritis. Finally, dermally applied copper complexes have been confirmed as pharmacoactive anti-inflammatory agents.

Clearly, various prior art approaches have been taken to employ copper as a means to directly alleviate the causes of inflammation and to promote tissue repair, which has led to have led to several improved copper compositions and dosage forms in an effort to maximize delivery of copper to the inflammatory areas. Examples of such delivery systems of the copper include parenteral (subcutaneous, intravascular, or intramuscular injection), oral, topical or inserts. The parenteral delivery of copper may be painful, inconvenient, require the presence of a physician, and cause further irritation at the site of injection. The oral delivery, on the other hand, often results in poorly absorbed copper by the gastric lining, thereby reducing their anti-inflammatory activity. Finally, the topical delivery of copper is commonly used when selecting a route in medicating inflammation such as, for example, arthritis. The administration of such topical dosage forms are patently desirable because of their unique and advantageous characteristics.

Notwithstanding the notoriety for topical dosage forms, many past and present topical copper complexes have not performed to their anticipated expectations as a means to effectively and conveniently treat inflammation or arthritis with copper. For example, the application of metal salts to proteinaceous membranes, such as skin, results in the attachment of the copper ions to the membrane components to form copper proteinates or salts. Thus, little if any copper ion, in the soluble, ionized state is ever introduced into the targeted inflammatory, for example, arthritic, areas. Further, copper salts can be corrosive to the skin possibly causing the patient to incur various types of lytic reactions. To overcome this undesirable characteristic, copper ions are complexed with a ligand or chelant to form a metal complex. That is, the copper is shielded from binding to the membrane components. An example of such topical complexes include copper-amine complexes and copper EDTA. Unfortunately, there are undesirable characteristics associated with these complexes which obviate their usefulness.

In U.S. Pat. No. 4,680,309 to the same inventor as the present invention, it is taught that tissue inflammation may be alleviated by delivering a metal complex consisting of a dialaki metal monoheavy metal chelate of an alpha or beta-hydroxy polycarboxlic acid. An example of the metal complex given is dialkalimetal monocopper (II) citrate.

Zinc ions are well known to have anti-viral activity. For example, the salt known as zinc acetate is used as a control substance in evaluating anti-viral compounds because zinc acetate is very toxic to viruses. However, such zinc salts have two inherent disadvantages that make them useless as therapeutic agents. In particular, the zinc salt is quite toxic to normal cells and it is very acidic. This makes it unsuitable for application to skin, much less mucus membranes. Further, because it is so acidic, about a pH of about 5, the zinc of zinc acetate is converted into an insoluble zinc oxide that has little or no anti-viral activity.

What is desired, however, is a means of having the advantages of both copper and zinc in a therapeutic compound without the disadvantages of each respective ion.

It has recently and surprisingly been discovered, though, that an admixture of the dialkalimetal monocopper (II) citrate with a zinc analog results in a dramatically improved anti-inflammatory treatment.

### SUMMARY

The present invention includes a palliative or therapeutic admixture for treating or medicating affected biological tissue in mammals. Such admixture is comprised of at least two monometal complexes, and each monometal complex is comprised of a multivalent metal and at least one polyfunctional organic ligand, wherein the ligand is in the form of an alkaline earth salt. The molar ratio of metal to ligand is 1:1.

The present invention further includes a method of treating or medicating affected biological tissue in mammals. The method comprises introducing to the affected biological tissue an effective amount of an admixture is comprised of at least two monometal complexes. Each monometal complex comprises a multivalent metal and at least one polyfunctional organic ligand. The ligand is in the form of an alkaline earth salt, and a molar ratio of metal to ligand is 1:1.

### DETAILED DESCRIPTION

In a preferred exemplary embodiment, the present invention is a palliative and/or therapeutic admixture for treating and/or medicating affected biological tissues in mammals.

Examples of affected biological tissues to which the various exemplary embodiments of the present invention may treat and medicate include infections and accompanying painful symptoms attributable and caused by Herpes viruses.

As an additional example, Aphthous ulcers of unknown etiology, or lesions associated with the frank suppression of the immune system by chemotherapeutic agents or by radiotherapy regimes have been surprisingly and unexpectedly reduced or even substantially eliminated by the use of the admixtures according to the various exemplary embodiments of the present invention.

Similarly, mucositis secondary to head/neck irradiation therapy and chemotherapies has been markedly reduced, as measured by reduction of xerostomia, commonly known as “dry mouth;” generation of increased flow to more fluid saliva; and by the marked reduction of tissue inflammation and concomitant pain which is often debilitating.

Possibly most surprisingly, admixtures according to the various exemplary embodiments of the present invention have been prepared and found to exhibit soothing, palliative properties and healing of tissues in a variety of medication-induced conditions, some of which have been exemplified above.

The admixture of the various exemplary embodiments of the present invention is comprised of two or more multivalent metals complexed with at least one polyfunctional organic ligand in the form of an alkaline earth salt in a mole ratio of metal to ligand as 1:1.

In a presently preferred form, the monometal complex of multivalent metal and a polyfunctional organic ligand in a ratio of 1:1 of the metal to the ligand has a dissociation property represented by a sigmoidally shaped plot on a pM-pH diagram. Specific examples of the metal complex are dialkali metal monocopper(II) citrates represented by disodium-, dipotassium- or dilithiummonocopper(II) citrate. These dialkali monocopper(II) citrates have a dissociation property represented by a sigmoidal plot, wherein the curve of two directions meet at a point within the pH range of about 7 to about 9. It has been established that these monocopper(II) complexes in basic media, on the order of about pH 9 to about 12, are very stable, i.e., have an effective stability constant, K_{eff}, of the order of about 10¹² to about 10¹³. However, K_{eff} of these monocopper(II) citrate complexes at a pH of about 7-9 are on the order of about 10⁵ to about 10¹². Therefore, at a pH of around 7, the effective stability constant of the monocopper(II) citrate complex is considerably lower (a thousand to a several hundreds of thousand times lower) and a significant free Cu⁺⁺ concentration is available for anti-inflammatory activity. For example, about 10% of the copper in the complex is in the ionized state at or about pH 7 while approximately 0.1% of the copper is ionized at or about pH 9.

Thus, it is to be understood that the anti-inflammatory complexes of this invention are sensitive to pH, and as the pH is lowered to or below about 7, copper ion is made more available. If tissue is intact, i.e., healthy without trauma, then there are few, if any, free endogenous reacting moieties to induce the dissociation of copper ions. If there is trauma caused by inflammation, then the copper ions are induced to dissociate and complex with the endogenous reacting moieties associated with such trauma, thereby reducing or alleviating the inflammation. In general, the complexes will then tend to dissociate over a pH range of about 3 to about 12. Above about pH 12, the complexes tend to be destroyed by the alkaline media, precipitating from the media as hydrous metal oxides. Below about pH 7, the instability of the metal complex results in high concentrations of the free Cu⁺⁺ upon demand, as explained to effect anti-inflammatory activities. At the pathological pH of about 7, below the skin, the controlled release is most effective. The complexes will preferably be dispersed in a vehicle to provide a composition having a pH of about 6.5 to about 9 for passage through the tissue upon typical administration to provide controlled release of the metal ions upon presentment of endogenous reacting moieties that are associated with inflammatory activities.

In accordance with this description and the presently preferred embodiment, it will become apparent that other metal complexes of polyfunctional organic ligands respond to the model of this invention where they exhibit the dissociation property characterized by a sigmoidal curve on a standard pM-pH diagram. For example, based upon the monometal-polyfunctional organic ligand complex of this invention, other metal ions of a monovalent or multivalent nature, specifically, divalent and polyvalent cations including zinc, nickel, chromium, bismuth, mercury, silver, cobalt, and other similar metallic or heavy metal cations may be employed. Other polyfunctional organic ligands may be substituted for the citric acid specifically exemplified by the preferred embodiment of this invention. Included among other polyfunctional ligands are the broader class of alpha or beta hydroxy polycarboxylic acids into which class the citric acid falls. Also, other functionally substituted acids such as alpha or beta amino, sulfhydro, phosphinol, etc., can be substituted in the molecular model of the metal complex of this invention and similar results can be achieved.

One particularly desirable metal complex in the 1:1 dialkali monometal polyfunctional organic ligand chelate family is disodium monocopper (II) citrate dihydrate, CAS Registry #65330-59-8. This material is sold under the tradename MCC™ by National Research Laboratories, Ltd. of Cincinnati, Ohio.

As set forth above and in the prior art, it is known that the use of compounds such as MCC and similar compounds singly may be used in a wide variety of utilities, including as antimicrobial agents. However, it has been surprisingly found that a mixture of two prior known such compounds exhibit unexpected synergistic results as a therapeutic in a wide range of applications, some of which are exemplified above.

For example, the prior art teaches using zinc with an amino acid in a ratio of 2:20, and an amount of copper present is 0.1 to 0.01% of the amount of zinc employed. In contrast, the various exemplary embodiments of the present invention utilize the salts of the 1:1 molar ratio complexes of divalent cations and polycarboxylic acids possessing unique pH dependent dissociation characters to deliver active ions in a physiological environment.

The unexpected results of using the admixtures according the various exemplary embodiments of the present invention are surprising, indeed. Whereas, for example, the MCC complex has demonstrated utility on relieving inflammatory processes, incorporating the zinc analog, disodium monozinc (II) citrate dihydrate, sold under the tradename MZC™ by National Research Laboratories, Ltd. of Cincinnati, Ohio, in a similar or reduced concentration in the therapeutic mixture results in dramatically improved results in anti-inflammatory performance.

The prior art teaches examples of a 1:1 complex similar to the various exemplary embodiments of the present invention, the complexes of the prior art include copper complexes and zinc complexes. The copper is present to counterbalance a large uptake of zinc. Large ingestion of zinc, e.g. 100 mg a day, may result in a depression of blood levels of the beneficial form of circulating protein known as high-density lipoprotein, commonly known as HDL. However, the copper of these prior art complexes have little to minimal therapeutic activity as contrasted to the 1:1 complexes of various metals and polyfunctional groups as in the present invention.

Further, the prior art teaches using simple salts rather than the complexes of the present invention. As such, the simple salts of the prior art possess no proton induced dissociation character and are barely ionized, much less water soluble at the physiological pH range from about 7 to less than about 8. The simple salts taught by the prior art, as exemplified by the zinc acetate description above, are essentially inert when compared to the high activity of the 1:1 complexes according to the various exemplary embodiments of the present invention.

Because of the synergistic effect of combining the 1:1 complexes according to the various exemplary embodiments of the present invention, the concentration of either of the complexes does not need to be increased, and in some cases can be decreased.

The admixture of the 1:1 complexes according to the various exemplary embodiments of the present invention can be in the form of a solid, liquid, gel, or foam.

While this invention has been described in conjunction with the specific embodiments outlined above, it is evident that many alternatives, modifications and variations will be apparent to those skilled in the art. Accordingly, the preferred embodiments of the invention as set forth above are intended to be illustrative, not limiting. Various changes may be made without departing from the spirit and scope of the invention

What is claimed is:

1. A palliative or therapeutic admixture for treating or medicating affected biological tissue in mammals, the admixture is comprised of effective amounts of at least two monometal complexes, each monometal complex comprising:
a multivalent metal; and
at least one polyfunctional organic ligand, wherein the ligand is in the form of an alkaline earth salt;
wherein a molar ratio of metal to ligand is 1:1.

2. The admixture according to claim 1, wherein the multivalent metal is selected from the group consisting of copper, zinc, nickel, chromium, bismuth, mercury, silver, and cobalt.

3. The admixture according to claim 1, wherein at least one monometal complex is disodium monocopper (II) citrate dihydrate (MCC).

4. The admixture according to claim 1, wherein at least one monometal complex is disodium monozinc (II) citrate dihydrate (MZC).

5. The admixture according to claim 1, wherein the admixture is in a form of a solid, liquid, gel, or foam.

6. The admixture according to claim 1, wherein the admixture has a pH of about 7.0 to less than about 8.0.

7. A method of treating or medicating affected biological tissue in mammals, comprising:
introducing to the affected biological tissue an effective amount of an admixture comprising at least two monometal complexes, each monometal complex comprising a multivalent metal and at least one polyfunctional organic ligand, wherein the ligand is in the form of an alkaline earth salt, and wherein a molar ratio of metal to ligand is 1:1.

8. The method according to claim 7, wherein the multivalent metal is selected from the group consisting of copper, zinc, nickel, chromium, bismuth, mercury, silver, and cobalt.

9. The method according to claim 7, wherein at least one monometal complex is disodium monocopper (II) citrate dihydrate (MCC).

10. The method according to claim 7, wherein at least one monometal complex is disodium monozinc (II) citrate dihydrate (MZC).

11. The method according to claim 7, wherein the affected biological tissues comprise skin.

12. The method according to claim 7, wherein the affected biological tissues developed due to infection of herpes virus, chemotherapeutic agents, radiotherapy regimes, or combination thereof.

13. The method according to claim 7, wherein the admixture has a pH of about 7.0 to less than about 8.0.

14. The method according to claim 7, wherein the admixture is a in a form of a solid, liquid, gel, or foam.
